(19) United States
(12) Patent Application Publication
(10) Pub. No.: US 2008/0003635 A1
(43) Pub. Date: Jan. 3, 2008

(54) High Throughput Collagen Synthesis Assay

(75) Inventors: Osman Nidai Ozes, San Bruno, CA (US); Lawrence M. Blatt, San Francisco, CA (US)

Correspondence Address: INTERMUNE, INC., 3280 BAYSHORE BLVD., BRISBANE, CA, 94005, US

(73) Assignee: InterMune, Inc., Brisbane, CA (US)

(21) Appl. No.: 11/761,500

(22) Filed: Jun. 12, 2007

Related U.S. Application Data
(60) Provisional application No. 60/804,551, filed on Jun. 12, 2006.

(51) Int. Cl. C12Q 1/02

(52) U.S. Cl. 435/29

(57) ABSTRACT

The present invention provides for a new plate-based high throughput screening of collagen synthesis wherein the collagen is left intact in the cells. The present invention also provides for collagen synthesis assay methods, methods of identifying candidate compounds which modulate collagen synthesis, and collagen synthesis assay compositions useful for modeling collagen depositions disorders, comprising idiopathic pulmonary fibrosis, liver fibrosis, renal fibrosis, heart fibrosis, rheumatoid arthritis, and atherosclerotic plaques.

### RELATED APPLICATIONS

This application claims the benefit under 35 U.S.C. §119(e) of U.S. Provisional Application No. 60/804,551, filed Jun. 12, 2006, which is incorporated herein by reference in its entirety.

### FIELD OF THE INVENTION

The present invention relates to assay methods and compositions useful for identifying candidate compounds which modulate collagen synthesis wherein the collagen is left intact.

### BACKGROUND OF THE INVENTION

Undesirable accumulation of collagen results in the development of numerous diseases, including kidney, heart, liver and pulmonary fibrosis, liver cirrhosis, and arthritic diseases. As such, there has been a growing interest in elucidating the regulation of collagen metabolism. Previous techniques for quantitating collagen synthesis were time-consuming and expensive: evaluating the uptake of radio-labeled proline, quantitation of hydroxyproline using HPLC, and precipitation of collagen using Sirius Red (Peterkofsky and Diegelmann. Biochemistry. 1971:10:988-994; Campa J S et al. Anal Biochem. 1990:186:257-263; Li Y Y et al. Proc Natl Acad Sci. 2000:97:12746-12751).

Radioactive collagen may be quantitatively measured by evaluating the uptake of radio-labeled [¹⁴C]proline into collagen using a liquid scintillation counter. While simple in theory, this method is time consuming, requiring nearly 24 hours of preparation, and expensive, due to the radioactive materials and equipment. Delays in the method also occur during the absorption of the [¹⁴C]proline into the cell, incorporation into collagen and secretion into the media before it can be measured.

Use of High-Pressure Liquid Chromatography (HPLC) to measure collagen requires isolation of hydroxyproline from the cells via derivation with NBD-Cl and reverse-phase HPLC. However, the light sensitivity of the derivatized amino acids prevents large batch runs and this method is not specific to the hydroxyproline in collagen alone—any hydroxyproline contained in the mixture will be measured. Although not time consuming as the previous method, the HPLC device is expensive.

Sirius Red which has sulphonic acid side chain groups which react with the side chain groups of the basic amino acids present in collagen. The Sircol® Soluble Collagen Assay involves using at least 3 different test tubes: (1) reagent blanks, (2) collagen standards and (3) the test samples. Sircol dye is added to all test tubes and centrifuged to pack the collagen-dye pellet at the bottom of the tubes. The supernatant is drained and the pellet is recovered using an alkali reagent to bring the collagen back into solution. Because of the alkali reagent, the collagen measurements must be taken within 3 hours. The measurements obtained from the reagent blank tubes must be subtracted from the test samples. Since the amount of dye required is related to the amount of collagen being measured, the experiment may need to be repeated until the correct amount of dye is used.

### SUMMARY OF THE INVENTION

The present invention provides for a new plate-based high throughput screening of collagen synthesis. In one embodiment, the invention provides for a collagen synthesis assay composition, comprising growing collagen producing cells to confluency in the presence of a candidate compound, a fixing agent, a solvent and a collagen complexing dye; determining whether the candidate compound modulates collagen synthesis and wherein the collagen is left intact in said cells.

In another embodiment, the invention provides for a collagen synthesis assay method, comprising intermixing collagen producing cells grown to confluency in the presence of a candidate compound, a fixing agent, a solvent and a collagen complexing dye; determining whether the candidate compound modulates collagen synthesis; and wherein the collagen is left intact in said cells.

In another embodiment, the invention provides for a collagen synthesis assay method, comprising intermixing collagen producing cells grown to post-confluency in the presence of a candidate compound, a fixing agent, a solvent and a collagen complexing dye; determining whether the candidate compound modulates collagen synthesis; and wherein the collagen is left intact in said cells.

In another embodiment, the invention provides for a collagen synthesis assay method, comprising intermixing collagen producing cells grown to confluency in the presence of a candidate compound, a fixing agent, a solvent and a collagen complexing dye; determining whether the candidate compound modulates collagen synthesis; and wherein the collagen is left intact in said cells, wherein said determination is made visually or colorimetrically using instruments selected from the group consisting of a spectrophotometer, colorimeter, spectroradiometer, densitometer, and microwell plate reader.

In another embodiment, the invention provides for a collagen synthesis assay method, comprising intermixing collagen producing cells grown to confluency in the presence of a candidate compound, a fixing agent, a solvent and a collagen complexing dye; determining whether the candidate compound modulates collagen synthesis; and wherein the collagen is left intact in said cells, wherein said determination is used as a model for collagen deposition disorders selected from the group consisting of idiopathic pulmonary fibrosis, liver fibrosis, renal fibrosis, heart fibrosis, rheumatoid arthritis, and atherosclerotic plaques.

In another embodiment, the invention provides for a collagen synthesis assay method, comprising intermixing collagen producing cells grown to confluency in the presence of a candidate compound, a fixing agent, a solvent and a collagen complexing dye; determining whether the candidate compound modulates collagen synthesis; and wherein the collagen is left intact in said cells, wherein said assay is used in a post-transplant patient.

In another embodiment, the invention provides for a method of identifying a candidate compound which modulates collagen synthesis, comprising growing collagen producing cells in the present of said candidate compound; assaying the mixture to determine whether the candidate compound modulates collagen synthesis; and wherein the collagen is left intact in said cells.

In another embodiment, the invention provides for a method of identifying a candidate compound which modulates collagen synthesis, comprising growing collagen producing cells to confluency in the presence of said candidate compound; assaying the mixture to determine whether the candidate compound modulates collagen synthesis; and wherein the collagen is left intact in said cells.

In another embodiment, the invention provides for a method of identifying a candidate compound which modulates collagen synthesis, comprising growing collagen producing cells to post-confluency in the presence of said candidate compound; assaying the mixture to determine whether the candidate compound modulates collagen synthesis; and wherein the collagen is left intact in said cells.

In another embodiment, the invention provides for a method of identifying a candidate compound which modulates collagen synthesis, comprising growing collagen producing cells to confluency in the presence of said candidate compound; assaying the mixture to determine whether the candidate compound modulates collagen synthesis; and wherein the collagen is left intact in said cells, wherein said determination is made visually or colorimetrically using instruments selected from the group consisting of a spectrophotometer, colorimeter, spectroradiometer, densitometer, and microwell plate reader.

In another embodiment, the invention provides for a method of identifying a candidate compound which modulates collagen synthesis, comprising growing collagen producing cells to confluency in the presence of said candidate compound; assaying the mixture to determine whether the candidate compound modulates collagen synthesis; and wherein the collagen is left intact in said cells, wherein said determination is used as a model for collagen deposition disorders selected from the group consisting of idiopathic pulmonary fibrosis, liver fibrosis, renal fibrosis, heart fibrosis, rheumatoid arthritis, and atherosclerotic plaques.

In another embodiment, the invention provides for a method of identifying a candidate compound which modulates collagen synthesis, comprising growing collagen producing cells to confluency in the presence of said candidate compound; assaying the mixture to determine whether the candidate compound modulates collagen synthesis; and wherein the collagen is left intact in said cells, wherein said assay is used in a post-transplant patient.

In another embodiment, the invention provides for a collagen synthesis assay composition, comprising growing collagen producing cells to post-confluency in the presence of a candidate compound, a fixing agent, a solvent and a collagen complexing dye; determining whether the candidate compound modulates collagen synthesis and wherein the collagen is left intact in said cells.

In another embodiment, the invention provides for a collagen synthesis assay composition, comprising growing collagen producing cells to confluency in the presence of a candidate compound, a fixing agent, a solvent and a collagen complexing dye; determining whether the candidate compound modulates collagen synthesis and wherein the collagen is left intact in said cells, wherein said determination is made visually or colorimetrically using instruments selected from the group consisting of a spectrophotometer, colorimeter, spectroradiometer, densitometer, and microwell plate reader.

In another embodiment, the invention provides for a collagen synthesis assay composition, comprising growing collagen producing cells to confluency in the presence of a candidate compound, a fixing agent, a solvent and a collagen complexing dye; determining whether the candidate compound modulates collagen synthesis and wherein the collagen is left intact in said cells, wherein said determination is used as a model for collagen deposition disorders selected from the group consisting of idiopathic pulmonary fibrosis, liver fibrosis, renal fibrosis, heart fibrosis, rheumatoid arthritis, and atherosclerotic plaques.

In another embodiment, the invention provides for a collagen synthesis assay composition, comprising growing collagen producing cells to confluency in the presence of a candidate compound, a fixing agent, a solvent and a collagen complexing dye; determining whether the candidate compound modulates collagen synthesis and wherein the collagen is left intact in said cells, wherein said assay is used in a post-transplant patient.

### DETAILED DESCRIPTION OF THE PREFERRED EMBODIMENTS

Assay Methods

An embodiment provides a collagen synthesis assay method, comprising intermixing collagen producing cells grown to confluency in the presence of a candidate compound, a fixing agent, a solvent and a collagen complexing dye; determining whether the candidate compound modulates collagen synthesis; and wherein the collagen is left intact in said cells. Preferentially, the cells are grown to post-confluency.

The intermixing may be conducted by mixing a candidate compound with collagen producing cells along with one or more of the various assay composition ingredients described above, preferably in the amounts described above and under the conditions described above to produce an assay composition.

In an embodiment, the assay method comprises determining whether the candidate compound modulates the collagen synthesis of the collagen producing cells. For example, in an embodiment, intermixing of the candidate compound and the collagen producing cells may enhance collagen synthesis in the collagen producing cells. In another embodiment, intermixing of the candidate compound and the collagen producing cells may inhibit collagen synthesis in the collagen producing cells. In another embodiment, intermixing of the candidate compound and the collagen producing cells may have no effect on collagen synthesis in the collagen producing cells.

Methods of Identifying Compounds Having Collagen synthesis Modulating Activity

An embodiment provides a method of identifying compounds having collagen synthesis modulating activity, comprising growing collagen producing cells to confluency in the presence of a candidate compound, assaying the mixture to determine whether the candidate compound modulates collagen synthesis wherein the collagen in left intact in the cells. In one embodiment, the candidate compound may enhance collagen synthesis in the collagen producing cells. In another embodiment, the candidate compound may inhibit collagen synthesis in the collagen producing cells. In another embodiment, the candidate compound may have no effect on collagen synthesis in the collagen producing cells.

In another embodiment, different wells on the same plate may contain a different mixture of collagen synthesis cells and candidate compounds to allow for high-throughput screening. In one embodiment, a 48-well plate may contain 1 control and 47 different combinations of collagen synthesis cells and candidate compounds. In another embodiment, a 48-well plate may contain 1 control and 7 different combinations of collagen synthesis cells, with the control and each combination repeated 6 times on the 48-well plate.

Various embodiments are described herein, including assay compositions and assay methods. In many cases the assay compositions may be used in the assay methods, and the assay methods may utilize the assay compositions. Thus, it will be understood that a particular description herein is not necessarily limited to the context of the embodiment in which it is set forth, and that it may apply to other embodiments. For example, aspects of the descriptions of the various ingredients of the assay compositions may apply to embodiments of the assay methods, and aspects of the descriptions of the mixing conditions for the assay methods may apply to embodiments of the assay compositions. However, the various embodiments described herein are not limited to use with other embodiments and thus, for example, the assay compositions embodiments may be utilized in other methods, and the assay method embodiments may utilize other assay compositions.

Assay Compositions

An embodiment provides an assay composition, comprising growing collagen producing cells to confluency in the presence of a candidate compound, a fixing agent, a solvent, and a collagen complexing dye. The candidate compound, or mixture of candidate compounds, is preferentially selected to modulate collagen synthesis. The collagen is preferably left intact in the collagen producing cells. The assay compositions may be used to determine whether the candidate compound, or candidate compounds, displays collagen synthesis modulating activity. For example, the assay compositions may be used in the assay methods described below in greater detail. In preferred embodiments, the assay compositions may be used in assay methods (including, but not limited to, those described herein) to determine whether a candidate compound has collagen synthesis modulating activity.

The assay composition comprises a candidate compound, or mixture of candidate compounds. The candidate compound, or mixture of candidate compounds, may be referred to herein as a “candidate compound.” The candidate compound is the subject of the assay, e.g., the assay is used to determine whether the candidate compound modulates collagen synthesis. For example, the collagen producing cells may be intermixed with pirfenidone or a pirfenidone analog, interferon alpha, interferon gamma, TGF-beta, or any combination of the above.

The assay composition also comprises collagen producing cells including, but not limited to, fibroblasts, myofibroblasts, fibrocytes, epithelial cells, endothelial cells, human synovial cells, liver stellate cells. The collagen producing cells are also the subject of the assay, e.g., the assay is used to determine whether the candidate compound enhances, inhibits or has no effect on the collagen synthesis of the cells.

The assay composition also comprises a fixing agent such as glutaraldehyde. Other fixing agents are well known in the art.

The assay composition also comprises a solvent. When determining the modulating activity on acid soluble collagen, the solvent is an acid such as acetic acid. When determining the modulating activity on salt soluble collagen, the solvent is a salt. When determining the modulating activity on pepsin soluble collagen, the solvent is a salt and pepsin. Such solvents are also well known in the art.

The assay composition also comprises a collagen complexing dye such as Sirius Red or dye methyl violet.

The various assay composition ingredients are preferentially selected to facilitate collagen synthesis modulating activity and may include additional ingredients beyond those described herein. Such compatibility may be determined by routing experimentation by those skilled in the art, in light of the guidance provided herein. For example, in an embodiment, a collagen synthesis assay composition may be performed with human lung fibroblasts, 1 mM pirfenidone, 5 ng/ml TGF-beta, 0.5% glutaraldehyde in PBS, ddH₂0, 0.5 M acetic acid and 300 μl of Sirius Red.

### EXAMPLE

Methods

Human lung fibroblast cells HFL1 were seeded as 5×10⁴ cells/well in 48-well plate and cultured for 3-7 days in FK12 medium supplemented with 10% heat inactivated fetal calf serum, 2 mM L-glutamine, streptomycin (100 μg/ml), and penicillin (100 units/ml). After the cells reached post-confluency, ascorbic acid (20 μg/ml), proline (10 μMol), and TGF-□ (250-1000 pg/ml) were added and cells were incubated for 72 hours. Medium was removed and cells were fixed to the plate with 0.5% glutaraldehyde, prepared in 1× phosphate-buffered-saline (PBS), for 30 minutes at room temperature. Glutaraldehyde was removed, cells were washed with ddH₂O and the first 24 wells, which were used to determine collagen synthesis, were treated with 0.5 M acetic acid for 30 minutes, while the second 24 wells were stained with 0.1% crystal violet for viability for 30 minutes at room temperature. After all of the wells were washed with ddH₂O, 250 μl of Sirius Red was added to acetic acid-treated cells for 2 hours. The Sirius Red was then removed, and the wells were washed with ddH₂O. Absorbed Sirius Red and crystal violet were eluted with 300 μl of alkaline solution and 0.1 M citric acid (pH: 4.2) prepared in 50% ethanol, respectively.

Results

Absorbances of eluted Sirius Red and crystal violet were determined at 540 and 600 nm. The extent of collagen accumulation was determined by normalizing the levels of induced collagen to cell density. Results were validated by quantifying the suppression of TGF-□ (T) induced collagen synthesis by 1 mM pirfenidone (P) and 300 pg/mL interferon gamma-1b (A) and comparing the results to those obtained using the conventional Sirius Red procedure.

TABLE 1
Fold Induction of Collagen at 1000 pg/ml TGF-β

| | Control | T | P | P + T | A | A + T | A + P | A + P + T |
|---|---|---|---|---|---|---|---|---|
| 24 hrs. | 1.000 | 1.111 | 0.998 | 1.077 | 1.077 | 1.076 | 1.026 | 1.019 |
| 48 hrs. | 1.000 | 1.126 | 0.954 | 1.119 | 1.035 | 1.102 | 0.960 | 1.058 |
| 72 hrs. | 1.000 | 1.500 | 0.917 | 1.101 | 0.897 | 0.991 | 0.971 | 1.108 |

TABLE 2
Fold Induction of Collagen at 500 pg/ml TGF-β

| | Control | T | P | P + T | A | A + T | A + P | A + P + T |
|---|---|---|---|---|---|---|---|---|
| 24 hrs. | 1.000 | 1.039 | 0.980 | 1.044 | 0.973 | 1.131 | 0.970 | 0.957 |
| 48 hrs. | 1.000 | 1.172 | 0.884 | 1.148 | 0.959 | 1.014 | 0.908 | 0.963 |
| 72 hrs. | 1.000 | 1.266 | 0.931 | 1.146 | 0.937 | 1.000 | 0.969 | 1.056 |

TABLE 3
Fold Induction of Collagen at 250 pg/ml TGF-β

| | Control | T | P | P + T | A | A + T | A + P | A + P + T |
|---|---|---|---|---|---|---|---|---|
| 24 hrs. | 1.000 | 1.096 | 0.973 | 1.025 | 1.047 | 1.079 | 0.993 | 0.983 |
| 48 hrs. | 1.000 | 1.102 | 0.997 | 1.069 | 1.040 | 1.120 | 0.957 | 1.062 |
| 72 hrs. | 1.000 | 1.130 | 0.928 | 1.028 | 0.897 | 1.005 | 0.959 | 1.037 |

What is claimed is:

1. A collagen synthesis assay method, comprising:
intermixing collagen producing cells grown to confluency in the presence of a candidate compound, a fixing agent, a solvent and a collagen complexing dye;
determining whether said candidate compound modulates collagen synthesis; and
wherein said collagen is left intact in said cells.

2. The method of claim 1, wherein said cells are grown to post-confluency.

3. The method of claim 1, wherein said determining is done visually or colorimetrically using instruments selected from the group consisting of a spectrophotometer, colorimeter, spectroradiometer, densitometer, and microwell plate reader.

4. The method of claim 1, wherein said assay is used as a model for collagen deposition disorders selected from the group consisting of idiopathic pulmonary fibrosis, liver fibrosis, renal fibrosis, heart fibrosis, rheumatoid arthritis, and atherosclerotic plaques.

5. The method of claim 1, wherein said assay is used in a post-transplant patient.

6. A method of identifying a candidate compound which modulates collagen synthesis, comprising:
growing collagen producing cells to confluency in the presence of said candidate compound;
assaying the mixture to determine whether said candidate compound modulates collagen synthesis; and
wherein said collagen is left intact in said cells.

7. The method of claim 6, wherein said cells are grown to post-confluency.

8. The method of claim 6, wherein said determining is done visually or colorimetrically using instruments selected from the group consisting of a spectrophotometer, colorimeter, spectroradiometer, densitometer, and microwell plate reader.

9. The method of claim 6, wherein said assay is used as a model for collagen deposition disorders selected from the group consisting of idiopathic pulmonary fibrosis, liver fibrosis, renal fibrosis, heart fibrosis, rheumatoid arthritis, and atherosclerotic plaques.

10. The method of claim 6, wherein said assay is used in a post-transplant patient.

11. A collagen synthesis assay composition, comprising:
growing collagen producing cells to confluency in the presence of a candidate compound, a fixing agent, a solvent, and a collagen complexing dye;
wherein said composition is useful in determining whether said candidate compound modulates collagen synthesis; and
wherein said collagen is left intact in said cells.

12. The method of claim 11, wherein said cells are grown to post-confluency.

13. The method of claim 11, wherein said determining is done visually or colorimetrically using instruments selected from the group consisting of a spectrophotometer, colorimeter, spectroradiometer, densitometer, and microwell plate reader.

14. The method of claim 11, wherein said assay is used as a model for collagen deposition disorders selected from the group consisting of idiopathic pulmonary fibrosis, liver fibrosis, renal fibrosis, heart fibrosis, rheumatoid arthritis, and atherosclerotic plaques.

15. The method of claim 11, wherein said assay is used in a post-transplant patient.
